(19) United States
(12) Patent Application Publication
(10) Pub. No.: US 2019/0054252 A1
(43) Pub. Date: Feb. 21, 2019

(54) OPTICAL METHODS FOR MEASURING DOSAGE IN A MEDICINE DELIVERY DEVICE

(71) Applicant: QUALCOMM Incorporated, San Diego, CA (US)

(72) Inventors: John Earl AMSCHLER, Del Mar, CA (US); Robert GANTON, San Diego, CA (US); Brian NIZNIK, San Diego, CA (US); Eugene DANTSKER, San Diego, CA (US); Paul Robert HOFFMAN, San Diego, CA (US)

(21) Appl. No.: 16/104,080

(22) Filed: Aug. 16, 2018

Related U.S. Application Data
(60) Provisional application No. 62/547,076, filed on Aug. 17, 2017.

(51) Int. Cl. A61M 5/315

(52) U.S. Cl. CPC A61M 5/31568; A61M 2205/3306; A61M 2205/3327; A61M 2205/3592; A61M 2205/3569; A61M 2205/3584; A61M 2205/3553

(57) ABSTRACT

Disclosed herein are techniques for drug delivery measurement. A medicine delivery device includes a cartridge for storing a medicine, a needle assembly coupled to a bottom of the cartridge, a piston located in the cartridge for pushing the medicine to dispense the medicine from the cartridge through the needle assembly, and a sensor configured to measure a distance between the piston and the bottom of the cartridge. The sensor includes a triangulation-based distance measurement unit, a resonant frequency-based distance measurement unit, a time-of-flight-based distance measurement unit, or a frequency-modulated continuous-wave time-of-flight-based distance measurement unit.

### CROSS-REFERENCE TO RELATED APPLICATIONS

This application claims the benefit of U.S. Provisional Application No. 62/547,076, filed Aug. 17, 2017, entitled “DRUG DELIVERY MEASUREMENT IN A MEDICINE DELIVERY DEVICE” which is incorporated herein by reference in its entirety.

### BACKGROUND

Techniques disclosed herein generally relate to medicine delivery devices, and more specifically to techniques for measuring the dose of a medicine contained in a medicine delivery device.

Certain medical devices may be used to deliver a medicine to a user. An example of such a medical device is an injection device (e.g., a syringe, an injector pen, a hypodermic needle, etc.). The injection device may hold the medicine in a fluid form (e.g., liquid, gas, etc.) in a drug container (e.g., a cartridge), and the injection device may include a variable dose setting mechanism (e.g., a dial or knob) which may allow a user to set a dose of the medicine to be dispensed. The variable dose setting mechanism may have numerical markings on the injection device that allows the user to set the proper dose. After setting the dose, the user may operate the injection device to deliver the set dose of the medicine into a patient's body manually or automatically.

Although an injection device with a dose setting mechanism may provide the user flexibility in setting a dose, the actual dose of the medicine being dispensed to the patient may be quite different from the desired dose (e.g., the dose set by the dose setting mechanism). For example, the variable dose setting mechanism may not have the desired resolution or accuracy; the dose accuracy in the injection device may be susceptible to the mechanical accuracy of the cartridge; temperature and/or pressure changes may affect the actual dose of the medicine being dispensed to the patient. The dosing error may have some adverse effects on the patient. As such, it is desirable to accurately measure out the actual dose of the medicine in the medicine delivery device before, during, and after a drug dispensing.

### SUMMARY

Embodiments disclosed herein use distance measurement techniques to help determine the actual dose of the medicine to be, being, or been dispensed to a patient from a medicine delivery device.

In certain embodiments, a medicine delivery device is disclosed. The medicine delivery device may include a cartridge for storing a medicine, a needle assembly coupled to a bottom of the cartridge, a piston located in the cartridge for pushing the medicine to dispense the medicine from the cartridge through the needle assembly, and a sensor configured to measure a distance between the piston and the bottom of the cartridge. In various implementations, the sensor may include one or more of a triangulation-based distance measurement unit, a resonant frequency-based distance measurement unit, a time-of-flight-based distance measurement unit, a frequency-modulated continuous-wave time-of-flight-based distance measurement unit, a light intensity-based distance measurement unit, an electrical impedance-based distance measurement unit, an electrical capacitance-based distance measurement unit, or a strain sensor-based distance measurement unit.

In some implementations of the medicine delivery device, the sensor may include a triangulation-based distance measurement unit. The triangulation-based distance measurement unit may include a light source configured to transmit a light beam to illuminate at least a portion of an object, a photodetector array comprising a plurality of photodetectors, and optics for forming an image of the illuminated portion of the object onto the photodetector array, wherein the image of the illuminated portion of the object illuminates a portion of the photodetector array at a given time. The triangulation-based distance measurement unit may be located at the bottom of the cartridge, in the piston, or at another location in the medicine delivery device.

In some implementations of the medicine delivery device, the sensor may include a resonant frequency-based distance measurement unit. In some implementations, the resonant frequency-based distance measurement unit may include a transmitter configured to generate a plurality of transmission signal pulses for transmitting towards the bottom of the cartridge. The plurality of transmission signal pulses, when reflected at interfaces between the cartridge and the stored medicine, may cause a formation of a standing wave signal inside the cartridge. The resonant frequency-based distance measurement unit may also include a receiver configured to measure the standing wave signal. In some implementations, the resonant frequency-based distance measurement unit may include an ultrasonic resonant frequency-based distance measurement unit.

In some implementations of the medicine delivery device, the sensor may include a time-of-flight-based distance measurement unit, such as an ultrasonic time-of-flight-based distance measurement unit. In some implementations, the time-of-flight-based distance measurement unit may include a frequency-modulated continuous-wave time-of-flight-based distance measurement unit. In some implementations, the frequency-modulated continuous-wave time-of-flight-based distance measurement unit may include a transmitter configured to generate a frequency-modulated continuous-wave signal, where a frequency of the frequency-modulated continuous-wave signal varies with time. The frequency-modulated continuous-wave time-of-flight-based distance measurement unit may also include a detector configured to measure a beat frequency between the generated frequency-modulated continuous-wave signal and a previously generated frequency-modulated continuous-wave signal returned from an object.

In certain embodiments, a method of drug delivery measurement is disclosed. The method may include measuring, before a drug dispensing, a first distance between a dispensing piston and a bottom of a cartridge of a medicine delivery device that stores a medicine, and determining a volume of the medicine in the cartridge based on the measured first distance. In some implementations, the method may also include measuring, during or after the drug dispensing, a second distance between the dispensing piston and the bottom of the cartridge, and determining a dispensing rate or a volume of the dispensed medicine based on the first distance and the second distance. In some embodiments, measuring the first distance may include measuring the first distance using one or more of a triangulation-based distance measurement unit, a resonant frequency-based distance measurement unit, a time-of-flight-based distance measurement unit, a frequency-modulated continuous-wave time-of-flight-based distance measurement unit, a light intensity-based distance measurement unit, an electrical impedance-based distance measurement unit, an electrical capacitance-based distance measurement unit, or a strain sensor-based distance measurement unit.

In certain embodiments, an apparatus may include means for performing one or more of the functions described in the present disclosure. In certain embodiments, a non-transitory computer-readable medium may have instructions embedded thereon for drug delivery measurement, the instructions including computer code for performing one or more of the functions described in the present disclosure. In certain embodiments, a system may include modules that respectively perform one or more of the functions described in the present disclosure.

Embodiments disclosed herein are also directed towards a medicine delivery device. In some embodiments, the medicine delivery device may include a shaft attached to a piston. The shaft may be configured to push the piston through a cartridge storing medicine to dispense the medicine from the cartridge through a needle assembly. In some embodiments, the medicine delivery device may include an optical distance measuring sensor configured to measure a distance between the piston and a surface of the cartridge opposite the piston.

In various embodiments, the optical distance measuring sensor may include a light source, optics, and a position sensitive detector. In various embodiments, the position sensitive detector is an array of pixels configured to detect light. In various embodiments, the optical distance measuring sensor is located at the bottom of the cartridge. In various embodiments, the light source is one of: a diode, an LED, or a laser. The light source may be configured to emit a light beam. In various embodiments, the light beam is collimated and sent towards the piston to illuminate at least a portion of the piston. In various embodiments, the optics are configured to collect reflected light from the illuminated portion of the piston and image the reflected light onto the position sensitive detector. In various embodiments, the reflected light from the illuminated portion of the piston forms a light spot on the position sensitive detector. The position sensitive detector may be an array of pixels configured to detect a position of the light spot. In various embodiments, a triangle is formed by a first line from the center of the optics to the center of the position sensitive detector and a second line from the center of the optics to the center of the light spot.

In various embodiments, the distance between the piston and the surface of the cartridge opposite the piston can be determined based on a distance between the light source and the piston. The distance between the light source and the piston may be determined based at least on a measured angle between the first line and the second line, and a distance between the light source and the optics.

Embodiments disclosed herein are also directed towards a method for measuring a distance between a piston of a medicine delivery device and a surface of a cartridge opposite the piston. In some embodiments, the method may include emitting a collimated light beam from a light source towards the piston. The light source may be located by the surface of the cartridge opposite the piston. In some embodiments, the method may include illuminating at least a portion of the piston. In some embodiments, the method may include collecting reflected light from the illuminated portion of the piston using optics located by the surface of the cartridge opposite the piston. In some embodiments, the method may include imaging, using the optics, the reflected light onto a position sensitive detector by the surface of the cartridge opposite the piston, in order to form a light spot on the position sensitive detector. In some embodiments, the method may include determining a distance between the piston and the surface of the cartridge opposite the piston based at least on relative positions of the optics, the position sensitive detector, and the light spot.

In various embodiments, the position sensitive detector is an array of pixels configured to detect light. In various embodiments, the surface of the cartridge opposite the piston is the bottom of the cartridge. In various embodiments, the light source comprises one of: a diode, an LED, or a laser. In various embodiments, the position sensitive detector is an array of pixels configured to detect a position of the light spot. In various embodiments, determining a distance between the piston and the surface of the cartridge opposite the piston further includes determining a center of the light spot and determining a measured angle between a first line and a second line. The first line is from the center of the optics to the center of the position sensitive detector. The second line is from the center of the optics to the center of the light spot. In various embodiments, determining a distance between the piston and the surface of the cartridge opposite the piston further includes determining a distance between the light source and the optics, and determining the distance between the piston and the surface of the cartridge based on the measured angle and the distance between the light source and the optics. In various embodiments, the light beam is in the infrared spectrum band. In various embodiments, the optics is a lens. In various embodiments, the emitted light beam is perpendicular to the surface of the cartridge opposite the piston.

Embodiments disclosed herein are also directed towards a medicine delivery device. In some embodiments, the medicine delivery device may include means for emitting a collimated light beam towards a piston. The piston may be configured to be pushed through a cartridge storing medicine to dispense the medicine from the cartridge. In some embodiments, the medicine delivery device may include means for illuminating at least a portion of the piston. In some embodiments, the medicine delivery device may include means for collecting reflected light from the illuminated portion of the piston at a location by the surface of the cartridge opposite the piston. In some embodiments, the medicine delivery device may include means for imaging the reflected light into a light spot. In some embodiments, the medicine delivery device may include means for determining the location of the light spot. In some embodiments, the medicine delivery device may include means for determining a distance between the piston and the surface of the cartridge opposite the piston based at least on the location of the light spot.

In various embodiments, the medicine delivery device may further include means for determining a center of the light spot. In various embodiments, the light beam is in the infrared spectrum band. In various embodiments, the emitted light beam is perpendicular to the surface of the cartridge opposite the piston.

Embodiments disclosed herein are also directed towards a non-transitory computer readable medium containing instructions. In some embodiments, the instructions, when executed by a processor, cause the processor to cause a light source to emit a collimated light beam from the light source towards a piston of a medicine delivery device. The light source may be located by a surface of a cartridge opposite the piston. The cartridge may store medicine. In some embodiments, the instructions, when executed by a processor, cause the processor to cause at least a portion of the piston to be illuminated. In some embodiments, the instructions, when executed by a processor, cause the processor to cause reflected light from the illuminated portion of the piston to be collected using optics located by the surface of the cartridge opposite the piston. In some embodiments, the instructions, when executed by a processor, cause the processor to cause the optics to image the reflected light onto a position sensitive detector by the surface of the cartridge opposite the piston, in order to form a light spot on the position sensitive detector. In some embodiments, the instructions, when executed by a processor, cause the processor to determine a distance between the piston and the surface of the cartridge opposite the piston based at least on relative positions of the optics, the position sensitive detector, and the light spot.

In various embodiments, the instructions, when executed by a processor, further cause the processor to determine a center of the light spot from a position of the light spot on the position sensitive detector. In various embodiments, the instructions, when executed by a processor, further cause the processor to determine a center of the position sensitive detector. In various embodiments, the instructions, when executed by a processor, further cause the processor to determine a center of the optics. In various embodiments, the instructions, when executed by a processor, further cause the processor to determine a measured angle between a first line and a second line. The first line may run from the center of the optics to the center of the position sensitive detector. The second line may run from the center of the optics to the center of the light spot. In various embodiments, the instructions, when executed by a processor, further cause the processor to determine a distance between the light source and the optics. In various embodiments, the instructions, when executed by a processor, further cause the processor to determine a distance between the light source and the piston based at least on the measured angle between the first line and the second line, and the distance between the light source and the optics. In various embodiments, the instructions, when executed by a processor, further cause the processor to determine the distance between the piston and the surface of the cartridge opposite the piston based at least on the distance between the light source and the piston.

This summary is neither intended to identify key or essential features of the claimed subject matter, nor is it intended to be used in isolation to determine the scope of the claimed subject matter. The subject matter should be understood by reference to appropriate portions of the entire specification of this disclosure, any or all drawings, and each claim. The foregoing, together with other features and examples, will be described in more detail below in the following specification, claims, and accompanying drawings.

### BRIEF DESCRIPTION OF THE DRAWINGS

Aspects of the disclosure are illustrated by way of example. In the accompanying figures, like reference numbers indicate similar elements.

FIG. 1 is a simplified diagram illustrating an example system for providing information about the dispensing of medicine by a medicine delivery device;

FIG. 2 is a simplified diagram illustrating an example medicine delivery device, according to certain embodiments;

FIG. 3 is a simplified block diagram illustrating example components of an example medicine delivery device as shown in FIG. 2, according to certain embodiments;

FIG. 4 is a simplified diagram illustrating the internal structure of an example medicine delivery device as shown in FIG. 2, according to certain embodiments;

FIG. 5 illustrates an optical distance/displacement measurement unit for measuring the volume of a medicine in a medicine delivery device, according to certain embodiments;

FIGS. 6A-6C illustrate an optical distance/displacement measurement unit for measuring the volume of a medicine in a medicine delivery device, according to certain embodiments;

FIG. 7 illustrates an ultrasonic distance/displacement measurement unit based on resonant frequency measurement for determining the volume of a medicine in a medicine delivery device, according to certain embodiments;

FIG. 8 illustrates an ultrasonic distance/displacement measurement unit based on time of flight measurement for determining the volume of a medicine in a medicine delivery device, according to certain embodiments;

FIG. 9 is a diagram illustrating time-of-flight-based short distance/displacement measurement using frequency-modulated continuous-wave, according to certain embodiments; and

FIG. 10 is a simplified flow chart illustrating an example method of drug delivery measurement, according to certain embodiments.

FIG. 11 is a simplified flow chart illustrating an example method of drug delivery measurement, according to certain embodiments.

### DETAILED DESCRIPTION

Several illustrative embodiments will now be described with respect to the accompanying drawings, which form a part hereof. The ensuing description provides embodiment(s) only, and is not intended to limit the scope, applicability or configuration of the disclosure. Rather, the ensuing description of the embodiment(s) will provide those skilled in the art with an enabling description for implementing an embodiment. It is understood that various changes may be made in the function and arrangement of elements without departing from the spirit and scope of this disclosure.

A medicine delivery device may include a dose setting mechanism to allow a user to set a volume of a medicine to be dispensed to a patient. The actual dose of the medicine being dispensed to the patient based on the volume set by the dose setting mechanism may be quite different from the desired dose dispensed to the patient. For example, the dose setting mechanism may not have the desired resolution or accuracy; the dose accuracy in the medicine delivery device may be susceptible to the mechanical accuracy of the cartridge; and temperature and/or pressure changes may affect the actual dose of the medicine being dispensed to the patient. The dosing error may have some adverse effects on the patient. As such, there is a need to accurately measure out the volume of the medicine in the medicine delivery device before, during, and/or after a drug dispensing.

Disclosed herein are techniques to facilitate dispensing of a correct dose of a medicine to a user. The techniques may include determining a volume of the medicine in a cartridge before, during, and/or after a drug dispensing based on, for example, the time of flight, triangulation, or cavity resonant frequency technique, and using, for example, optical (e.g., infrared), ultrasonic, or radio frequency signals.

I. System

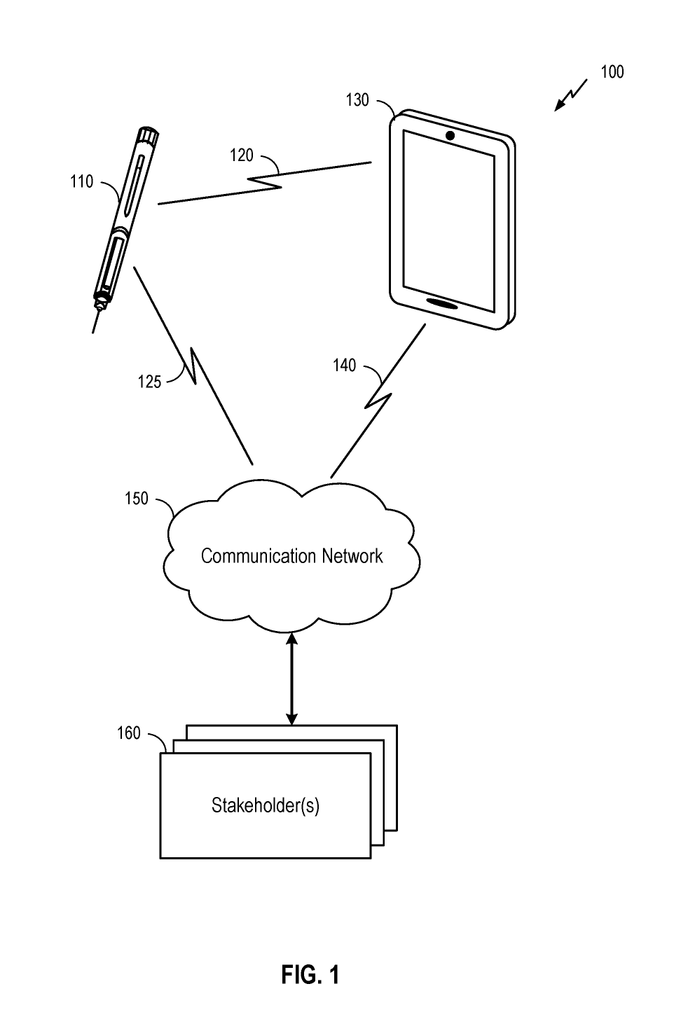

FIG. 1 is a simplified diagram illustrating an example system 100 for providing information about the dispensing of medicine by a medicine delivery device 110 to one or more stakeholder(s) 160. System 100 may comprise medicine delivery device 110 as described herein, along with a connecting device 130, communication network 150, and the stakeholder(s) 160. Medical delivery device 100 may be a device configured to deliver a medicine as a fluid (e.g., in a liquid form). It will be understood, however, that embodiments of a system 100 may include a different configuration of components, the addition and/or omission of various components, and/or the like, depending on desired functionality. Moreover, it will be understood that techniques described herein may be utilized in a medicine delivery device 110 that may not necessarily be part of a larger system, such as the system 100 illustrated in FIG. 1.

Medicine delivery device 110, which is described in more detail herein below, may be used to dispense a medicine to a patient. In the example of FIG. 1, medicine delivery device 110 may be an injection device such as, for example, a syringe, an injection pen, etc. A person (e.g., a doctor, nurse, or patient him/herself) may dispense the medicine by engaging a physical mechanism (e.g., pressing down on a plunger, actuating automatic injection, etc.). Through the physical mechanism, a dose of the medicine may be injected into the patient's tissue via a needle of medicine delivery device 110 inserted through the patient's skin into underlying tissue. In some embodiments, after the medicine is dispensed, medicine delivery device 110 may then register, store, and transmit data associated with the dispensing of the medicine to connecting device 130. The data may be transmitted wirelessly via a wireless communication link 120, using any of a variety of wireless technologies as described in further detail below. The dosage information may be transmitted to a drug adherence or drug compliance system that monitors the drug dosage activity of the user. The drug dosage information may be transmitted to the drug adherence or drug compliance system alone or in combination with other information such as time of drug delivery, identity of the user, route of drug delivery and/or identity of the drug being delivered, in order to form a record of the users drug or medicine usage. This record of the user's actual drug or medicine usage may be compared to a desired drug or medicine regimen to determine compliance or adherence to that regimen. That said, some embodiments may additionally or alternatively utilize wired communication or locally store the information in memory to be later retrieved.

Connecting device 130 may comprise any of a variety of electronic devices capable of receiving information from medicine delivery device 110 and communicating information to stakeholder(s) 160 via communication network 150. Connecting device 130 may include, for example, a mobile phone, tablet, laptop, portable media player, personal computer, or similar device. In some embodiments, connecting device 130 may comprise a specialized device for conveying information from medicine delivery device 110 (and possibly other medical devices) to stakeholder(s) 160. In some embodiments, connecting device 130 may comprise a device owned and operated by the patient (e.g., the patient's mobile phone). In other embodiments, the connecting device 130 may be owned and/or operated by another entity, such as a healthcare provider, insurance company, government agency, etc.

Connecting device 130 may execute an application to provide the data processing and/or relaying functionality illustrated in FIG. 1. In some embodiments, the application may be configurable by a user, or may be downloaded to connecting device 130 and executed automatically. The application may help to establish communication link 120 between medicine delivery device 110 and connecting device 130, which may or may not require input from the user, depending on the desired functionality. In some embodiments, the application may provide instructions to a user on the proper use of medicine delivery device 110 and/or provide feedback to a user about the detected use of medicine delivery device 110. Medicine delivery device 110 may also detect the dose set by the patient and transmit information related to the detected dosage to connecting device 130 (e.g., such as whether an incorrect dosage is set), as part of the feedback. Additional and/or alternative functionality of an application executed by connecting device 130 may be utilized as desired, such as, for example, relaying of the data to a remote destination, interacting with the patient about the medicine dispensing, etc.

Communication network 150 may comprise any of a variety of data communication networks, depending on the desired functionality. Communication network 150 may include any combination of radio frequency (RF), optical fiber, satellite, and/or other wireless and/or wired communication technologies. In some embodiments, communication network 150 may comprise the Internet and/or different data networks that may comprise various network types, including cellular networks, Wi-Fi® networks, etc. These network types may include, for example, a Code Division Multiple Access (CDMA) network, a Time Division Multiple Access (TDMA) network, a Frequency Division Multiple Access (FDMA) network, an Orthogonal Frequency Division Multiple Access (OFDMA) network, a Single-Carrier Frequency Division Multiple Access (SC-FDMA) network, a WiMax (IEEE 802.16), and so on. A CDMA network may implement one or more radio access technologies (RATs) such as cdma2000, Wideband-CDMA (W-CDMA), and so on. Cdma2000 includes IS-95, IS-2000, and/or IS-856 standards. A TDMA network may implement Global System for Mobile Communications (GSM), Digital Advanced Mobile Phone System (D-AMPS), or some other RAT. An OFDMA network may employ LTE (including LTE category M (CatM) or 5G), LTE Advanced, and so on. LTE, LTE Advanced, GSM, and W-CDMA are described in documents from 3GPP. Cdma2000 is described in documents from a consortium named “3rd Generation Partnership Project 2” (3GPP2). 3GPP and 3GPP2 documents are publicly available. The communication network 150 may additionally or alternatively include a wireless local area network (WLAN), which may also be an IEEE 802.11x network, and a wireless personal area network (WPAN) may be a Bluetooth network, an IEEE 802.15x, Zigbee® network, and/or some other type of network. The techniques described herein may also be used for any combination of wireless wide area network (WWAN), WLAN and/or WPAN.

Communication link 140 between connecting device 130 and communication network 150 may vary, depending on the technologies utilized by these components of the system 100. For embodiments where connecting device 130 is a smart phone capable of connecting with a cellular network and/or a Wi-Fi® network, communication link 140 may comprise a wireless communication link utilizing the mobile phone's cellular or Wi-Fi® functionality. In embodiments where connecting device 130 is a personal computer, communication link 140 may comprise a wired communication link that accesses communication network 150 via a cable or digital subscriber line (DSL) modem.

It is noted that some embodiments may not utilize a connecting device 130 to relay data to the communication network 150. In such embodiments, medicine delivery device 110 may connect directly to communication network 150 (as shown in FIG. 1 by communication link 125, which may be used in addition to, or as an alternative to, communication link 120). For example, medicine delivery device 110 may comprise a Long Term Evolution (LTE) category M (Cat-M) device, NarrowBand IoT (NB-IoT), or other Low Power Wide Area Network (LPWAN). Additionally or alternatively, medicine delivery device 110 may comprise wireless technology similar to the corresponding functionality of connecting device 130 described above. In such embodiments, the communication network may additionally or alternatively comprise a Bluetooth Mesh network (such as CSRMesh), a WiFi network, Zigbee, or WWAN (such as LTE, including Cat-M, or 5G). In some embodiments, medicine delivery device 110 may connect both with communication network 150 via communication link 125 and with connecting device 130 via communication link 120. In such embodiments, connecting device 130 may not need to separately communicate information regarding medicine delivery device 110 to stakeholders 160, but instead medicine delivery device 110 may communicate this information directly to stakeholders 160 via communication network 150. In some implementations, connecting device 130 may not be owned by the user. Rather, it may be a doctor's device, owned by an insurance company, etc.

As noted above, stakeholder(s) 160 may include any of a variety of entities with an interest in the proper dispensing of medicine by medicine delivery device 110. This may include an individual practitioner (e.g., a doctor or nurse), a hospital, a drug manufacturer, an insurance provider (or other payer), a government agency or other health organization, and/or the like. In some embodiments, the user of medicine delivery device 110 (e.g., the patient) may also be a stakeholder 160 to which information regarding the use of medicine delivery device 110 is provided. Governmental health regulations and/or legal agreements between the patient and/or stakeholder(s) 160 may apply to the dissemination of information regarding the dispensing of a drug by medicine delivery device 110 to stakeholder(s) 160.

II. Medicine Delivery Device (Injector Pen)

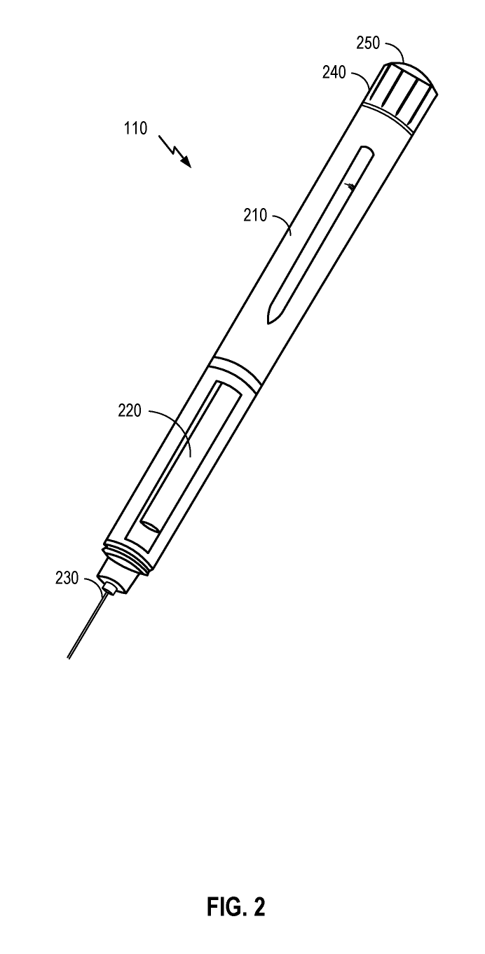

FIG. 2 is a simplified diagram illustrating an example medicine delivery device 110, according to certain embodiments. Medicine delivery device 110 may include a body 210 that may house dose dispensing and dose control mechanisms, including electrical and mechanical components. Mechanical components of a dose dispensing mechanism may include a movable component (e.g., a piston) controlled by the dose control mechanism and configured to displace a volume of the medicine through the reservoir chamber 220 (also referred to as a cartridge or vial) and out of a needle assembly 230. Embodiments of medicine delivery device 110 may further include a dose knob 240 (also referred to as a dial) that may be adjusted (e.g., by turning the knob clockwise or counterclockwise) to alter the dose to be dispensed by medicine delivery device 110. The dose may be dispensed by pressing dose dispensing button 250, which may be coupled to a dose dispensing mechanism to control the dispensing of the medicine.

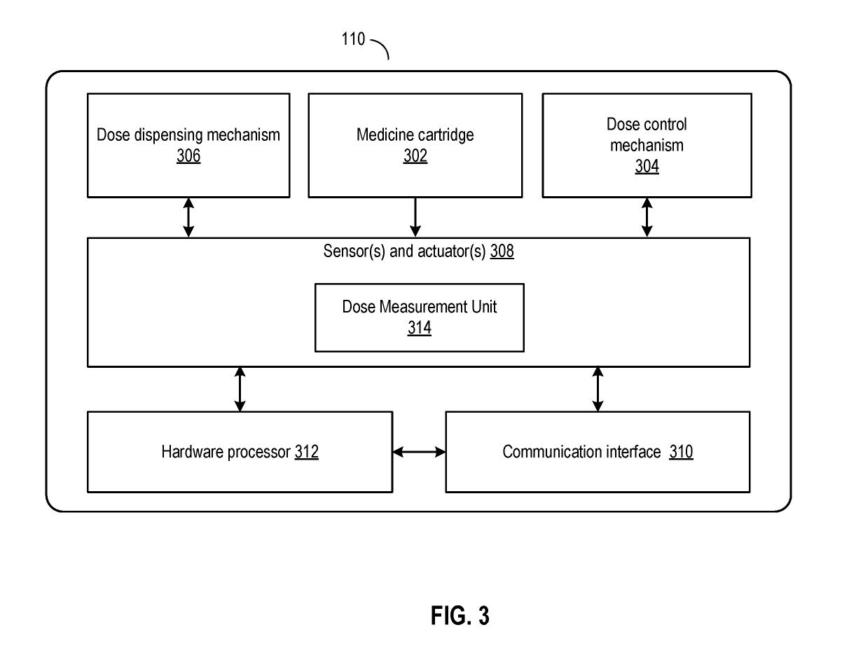

It will be understood, however, that medicine delivery device 110 illustrated in FIG. 2 is provided as a non-limiting example, according to an embodiment. Alternative embodiments may vary in size, shape, and/or other ways. A medicine delivery device 110 may be described more generally as having various components as illustrated in FIG. 3.

FIG. 3 is a simplified block diagram illustrating example components of an example medicine delivery device 110 as shown in FIG. 2, according to certain embodiments. Medicine delivery device 110 may include a housing (not shown) structured to hold a medicine cartridge 302, which may store a medicine to be dispensed by medicine delivery device 110. Medicine delivery device 110 may also include a dose control mechanism 304 to select or set a dose of the medicine to be dispensed. For example, dose control mechanism 304 may include a piston to set a volume of the medicine held within medicine cartridge to be dispensed. Medicine delivery device 110 may further include a dose dispensing mechanism 306 for dispensing a dose of the medicine from medicine cartridge 302, based on the dose selected or set by dose control mechanism 304.

Medicine delivery device 110 may include other devices to facilitate the dispensing of medicine. In the example of FIG. 3, medicine delivery device 110 may include sensor(s) and actuator(s) 308, and a hardware processor 312. Sensor(s) and actuator(s) 308 may include sensors and actuators to control the operations of the actuators based on the information collected by the sensors. For example, the sensors of sensor(s) and actuator(s) 308 may collect information of certain physical conditions at, for example, medicine cartridge 302, dose control mechanism 304, and dose dispensing mechanism 306. Based on the collected information, hardware processor 312 may control the actuators of sensor(s) and actuator(s) 308 to change the operations of dose control mechanism 304 and/or dose dispensing mechanism 306. For example, as to be described in more details below, the sensors of sensor(s) and actuator(s) 308 may include a dose measurement unit 314 for measuring the volume of the medicine in the cartridge at any given time, such as before, during, or after a drug dispensing using medicine delivery device 110. Based on the collected data, the hardware processor 312 may determine whether a correct dose has been dispensed. If the hardware processor 312 determines that the dose is not sufficient, the hardware processor 312 may control the actuator of sensor(s) and actuator(s) 308 to, for example, dispense additional dose of the medicine to the user.

Moreover, medicine delivery device 110 may include a communication interface 310 which may communicate using wireless and/or wired means (e.g., via wireless communication link 120 and/or 125 of FIG. 1). Communication interface 310 may enable transmission of information related to dispensing the drug. For example, the communication interface 310 may enable transmission of information indicating a dose set by the user and, in the event that an incorrect dosage is set, may enable transmission of a warning to the user about the incorrect dosage. The information may then be displayed to the user via an user interface, to assist the user in dispensing of the medicine. Moreover, as part of an interactive process, the communication interface 310 may also receive information related to a confirmation (or an overriding command) from the user that the medicine is to be dispensed according to the set dose. Communication interface 310 may relay the confirmation or overriding command to sensor(s) and actuator(s) 308, to enable dose dispensing mechanism 306 to dispense the medicine.

Although not shown in FIG. 3, medicine delivery device 110 may further include one or more non-transitory storage devices including, for example, a solid-state storage device, such as a random access memory (“RAM”), and/or a read-only memory (“ROM”), which may be programmable, flash-updateable and/or the like. Such storage devices may be configured to implement any appropriate data stores, including without limitation, various file systems, database structures, and/or the like. A set of instructions and/or code might be stored on a non-transitory computer-readable storage medium, which may then be executed by hardware processor 312 to perform the operations described above and operations as to be described below.

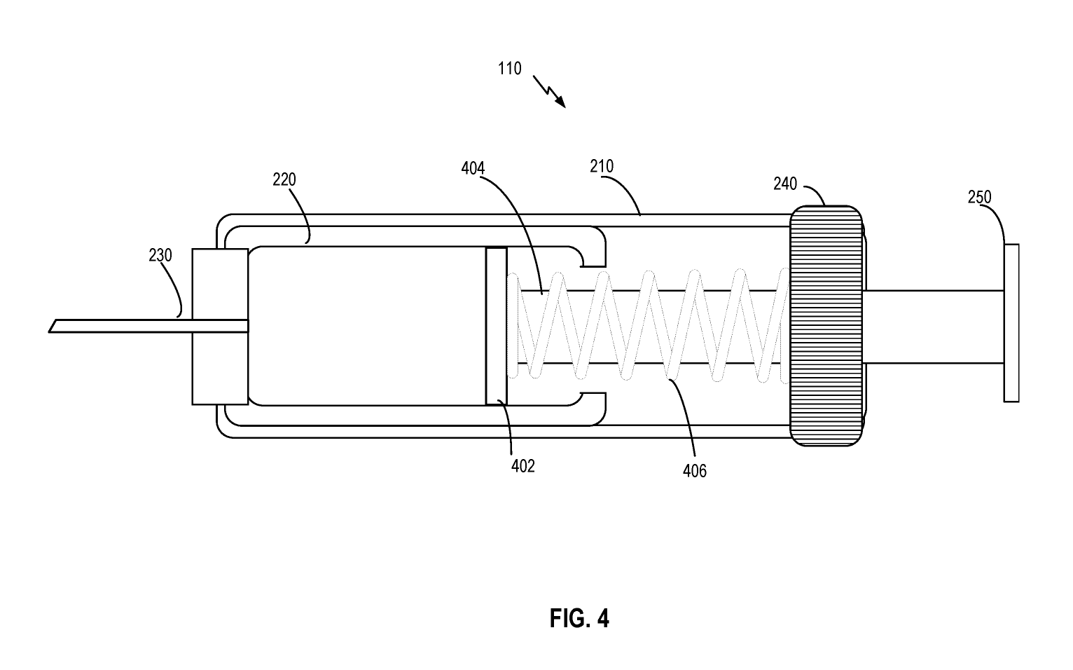

FIG. 4 is a simplified diagram illustrating the internal structure of an example medicine delivery device 110 as shown in FIG. 2, according to certain embodiments. It will be appreciated by a person of ordinary skill in the art that the illustration is not to scale, and the various components illustrated may vary in size, shape, arrangement, etc., as desired. As shown in FIG. 4, medicine delivery device 110 may include a dispensing piston 402. Dispensing piston 402 may be coupled to dose dispensing button 250 via a shaft 404. A user may rotate dose knob 240 to set the volume of the medicine to be dispensed. After the user selects the dose, the user may push down dose dispensing button 250, which may then push dispensing piston 402 towards needle assembly 230 to push the medicine out of reservoir chamber 220. In some implementations, medicine delivery device 110 may include a spring 406, which may be released when dose dispensing button 250 is pressed, such that spring 406 may automatically push dispensing piston 402 towards needle assembly 230, and may retract the needle from the patient after the injection.

In some implementations, body 210 may also hold various electronic units (not shown). The electronic units may include sensors, actuators, and processors of sensor(s) and actuator(s) 308. In some embodiments, the electronic units may include actuators to lock dispensing piston 402 (and/or shaft 404) at a fixed position to prevent dispensing piston 402 from pushing the medicine out of reservoir chamber 220. Moreover, the electronic units may include communication interface circuitries of communication interface 310. The communication interface circuitries may transmit information about the dosage setting, and receive a confirmation or overriding command to release the locking of dispensing piston 402 (and/or shaft 404), as described above.

One important aspect for making sure that the right dose of the right drug is dispensed to the right patient at the right time via the right route, is to accurately measure the volume of the medicine in the cartridge to be dispensed or the volume of the medicine that has been dispensed from the cartridge of a medicine dilivery device. Example embodiments of various techniques for measuring the volume of the medicine in the cartridge to be dispensed, or various techniques for measuring the volume of the medicine that has been dispensed from the cartridge are described in details below. In general, the volume of the medicine in the cartridge to be dispensed or the volume of the medicine that has been dispensed from the cartridge may be measured by measuring the distance or displacement of the dispensing piston from the bottom of the cartridge that is coupled to the needle assembly. For example, the distance between the dispensing piston and the bottom of the cartridge may be used to determine the volume of the medicine in the cartridge. The displacement of the dispensing piston before, during, or after a drug adminstration may be used to determine the volume of the medicine that has been dispensed during the drug dispensing.

III. Triangulation-Based Measurement

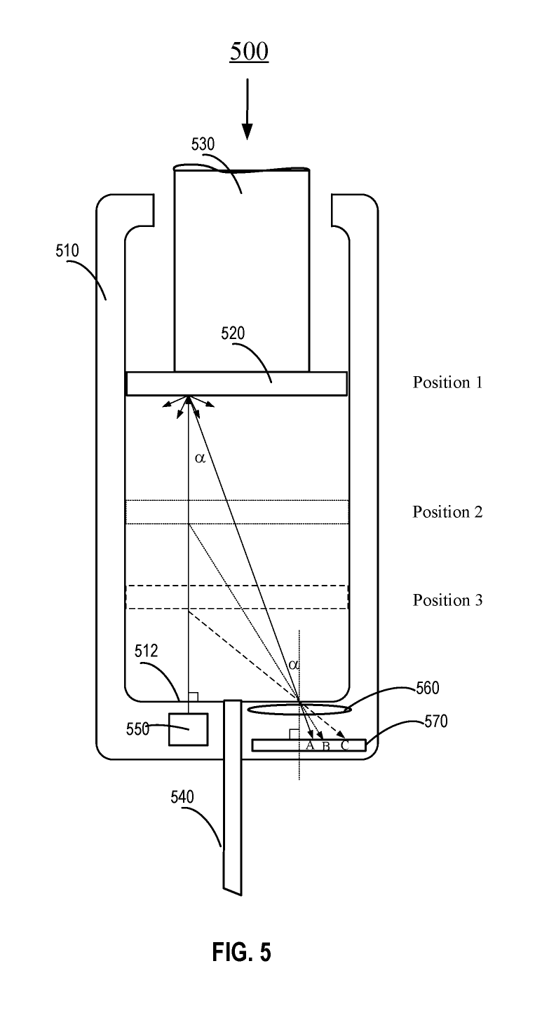

FIG. 5 illustrates an optical distance/displacement measurement unit for measuring the volume of the medicine in a medicine delivery device 500, according to certain embodiments. As medicine delivery device 110 described above with respect to FIG. 4, medicine delivery device 500 may include a cartridge 510 containing medicine to be dispensed to a patient (i.e., a drug container or vial), a dispensing piston 520 coupled to a shaft 530 (which may be coupled to a dose dispensing button, which is not shown in FIG. 5), and a needle assembly 540. A user may push the dose dispensing button to dispense the medicine from cartridge 510 to a patient through needle assembly 540.

Medicine delivery device 500 may also include an optical distance/displacement measurement unit for measuring the volume of the medicine in medicine delivery device 500. The optical distance/displacement measurement unit may include a light source 550 (e.g., an infrared LED or laser), optics 560, and a position sensitive detector (PSD) 570 (e.g., a pixel array). In the example shown in FIG. 5, the optical distance/displacement measurement unit may be located at the bottom of cartridge 510. It is noted that, in other embodiments, the optical distance/displacement measurement unit may be located at other parts of medicine delivery device 500.

In some embodiments, light source 550 may include a diode, an LED or a laser, and it may emit a light beam, such as an invisible light beam in the infrared spectrum band. The light beam may be collimated and sent towards dispensing piston 520 to illuminate at least a portion of dispensing piston 520. The reflected light from the illuminated portion of dispensing piston 520 may be collected by optics 560 and imaged onto PSD 570. In other words, the illuminated portion of dispensing piston 520 may be imaged by optics 560 (which may function as a camera lens) onto PSD 570. PSD 570 may include an array of pixels that may detect light. The locations of the pixels that detect the image (e.g., a light spot) of the illuminated portion of dispensing piston 520 formed by optics 560 may indicate the position of the image on position sensitive detector 570. Light source 550, PSD 570, and the illuminated portion of dispensing piston 520 may form a triangle. If the emitted light beam is perpendicular to the line formed by light source 550 and PSD 570, a right angle triangle may be formed.

As also shown in FIG. 5, a similar triangle may be formed by the center of optics 560, the center of PSD 570, and the light spot on PSD 570. By measuring the exact location of the light spot on PSD 570, angle α may be determined based on the distance between optics 560 and PSD 570. Based on the measured angle α and known distance between light source 550 and optics 560, the distance between light source 550 and dispensing piston 520 may be determined. The distance measurement resolution of the optical distance/displacement measurement unit may depend on the size of the light beam illuminating dispensing piston 520 and the pixel size of PSD 570.

The optical distance/displacement measurement unit may continuously measure the distance between dispensing piston 520 and the inner bottom 512 of cartridge 510 before, during, and/or after a drug dispensing. For example, at time t1, dispensing piston 520 may be at position 1, and the light spot on PSD 570 may be at location A. At time t2, dispensing piston 520 may be at position 2, and the light spot on PSD 570 may be at location B. At time t3, dispensing piston 520 may be at position 3, and the light spot on PSD 570 may be at location C. A change in measured distance between two measuring time instants may indicate the volume of the medicine being dispensed between the two measuring time instants, and hence the dispensing rate. A change in measured distance before and after a drug dispensing may indicate the total volume of the medicine being dispensed to the patient.

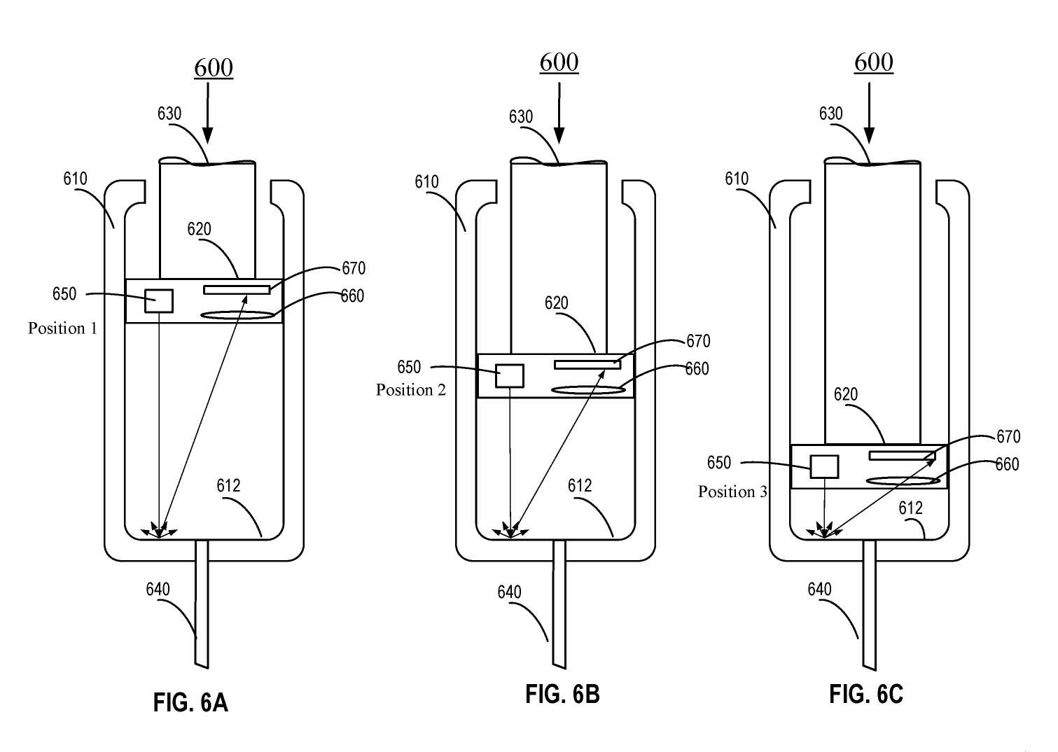

FIGS. 6A-6C illustrate an optical distance/displacement measurement unit for measuring the volume of the medicine in a medicine delivery device 600, according to certain embodiments. The optical distance/displacement measurement unit for measuring the volume of the medicine in medicine delivery device 600 may function similarly as the distance/displacement measurement unit described above with respect to FIG. 5. Medicine delivery device 600 may include a cartridge 610 containing a medicine to be dispensed to a patient, a dispensing piston 620 coupled to a shaft 630 (which may be coupled to a dose dispensing button not shown in FIG. 6), and a needle assembly 640. A user may push the dose dispensing button to dispense medicine from cartridge 610 to a patient through needle assembly 640. The optical distance/displacement measurement unit may include a light source 650 (e.g., an infrared LED or laser), optics 660, and a position sensitive detector (PSD) 670 (e.g., a pixel array). In the example shown in FIG. 6, the optical distance/displacement measurement unit may be located in dispensing piston 620 or shaft 630. Thus, light source 650 may emit a light beam towards a bottom 612 of cartridge 610 and optics 660 may image the illuminated portion of bottom 612 of cartridge 610 onto PSD 670.

As shown in FIG. 6A, at time t1, dispensing piston 620 may be at position 1, and the light spot on PSD 670 may be at one location. At time t2 (shown by FIG. 6B), dispensing piston 620 may be at position 2, and the light spot on PSD 670 may be at a second location. At time t3 (shown by FIG. 6C), dispensing piston 620 may be at position 3, and the light spot on PSD 670 may be at a third location. Thus, a change in the location of the light spot on PSD 670 may indicate a displacement of dispensing piston 620 within cartridge 610, which may in turn indicate the volume of the medicine being dispensed when dispensing piston 620 is pushed from one position to another.

IV. Resonant Frequency-Based Measurement

When wave signals (for example, acoustic signals such as ultrasonic signals) travel through a medium (i.e., traveling waves), they may be observed as waves with crests followed by troughs over a period of time. However, when the ultrasonic signals are incident on a mismatched boundary, the ultrasonic signals may be partially transmitted into the adjacent medium and partially reflected backwards, where the amount of reflection may be a function of the materials on the two sides of the boundary. For example, if a wave signal is traveling through a substantially solid medium and the adjacent medium is air, most of the wave signal may be reflected back into the solid medium due to the high level of impedance mismatch. On the other hand, when a wave signal is traveling through a first medium and the adjacent second medium is a medium having similar characteristics as the first medium, most of the wave signal may be transmitted into the second medium due to the close match. In any case, the reflected portion of the wave signal may interfere with consecutively generated wave signals in a given medium (e.g., a liquid) and produce an accumulated wave that may amplify over time, by the constructive interference of the plurality of signals over time.

With a proper selection of the excitation frequency for a given material and thickness, the transmitted signal (e.g., generated ultrasonic wave signal) and the reflected signal may interact in such a manner so as to constructively overlap with each other as they bounce between the boundaries of the medium, causing the ultrasonic wave to appear standing, which may be referred to as a standing wave, standing wave signal, or ultrasonic standing wave signal. Furthermore, with continued generation and application of the excitation signal pulses, the constructive transmitted and reflected signals may continue to add up in amplitude until an equilibrium value is approached. Thus, the medium and its adjacent mediums may form an acoustic cavity that exhibits resonance or resonant behavior for forming the standing wave signal at a particular frequency. An acoustic cavity may also be interchangeably referred to as an acoustic resonant cavity, a resonant acoustic cavity, a resonant cavity, an acoustic resonator or a cavity resonator, without deviating from the scope of the invention. An acoustic resonant cavity may have more than one resonant frequency. The resonant frequency may depend on the characteristics of the medium and the length of the cavity. Thus, the length of the resonant cavity may be determined based on the resonant frequency.

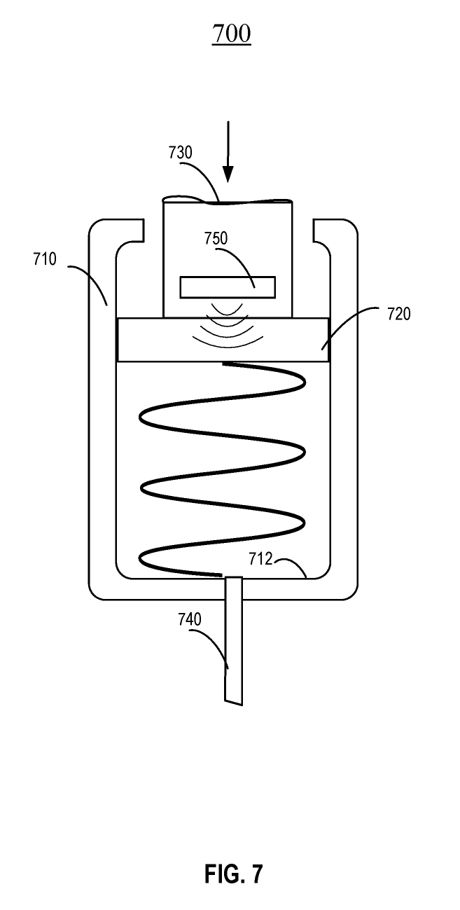

FIG. 7 illustrates an ultrasonic distance/displacement measurement unit based on resonant frequency measurement for determining the volume of a medicine in a medicine delivery device 700, according to certain embodiments. Medicine delivery device 700 may include a cartridge 710 containing a medicine to be dispensed to a patient, a dispensing piston 720 coupled to a shaft 730 (which may be coupled to a dose dispensing button not shown in FIG. 7), and a needle assembly 740. A user may push the dose dispensing button to dispense the medicine from cartridge 710 to a patient through needle assembly 740.

Medicine delivery device 700 may also include an ultrasonic distance/displacement measurement unit 750. Ultrasonic distance/displacement measurement unit 750 may be located in dispensing piston 720 or shaft 730. Ultrasonic distance/displacement measurement unit 750 may include an ultrasonic transmitter for generating ultrasonic signals at different frequencies and transmitting the ultrasonic signals through dispensing piston 720 towards the liquid medicine in cartridge 710. Ultrasonic distance/displacement measurement unit 750 may also include an ultrasonic receiver for measuring the returned ultrasonic signals or the ultrasonic signals at the boundary between the liquid medicine in cartridge 710 and dispensing piston 720 (or a bottom 712 of cartridge 710).

In some implementations, the ultrasonic transmitter of ultrasonic distance/displacement measurement unit 750 may gradually change the frequency of the transmitted ultrasonic signals until a resonant condition is detected. The frequency of the transmitted ultrasonic signals at the resonant condition may then be used to determine the length of the resonant cavity (i.e., the distance between piston 720 and bottom 712 of cartridge 710).

Even though ultrasonic signals are used in the above-described embodiment, a person skilled in the art would appreciate that other signals, such as optical signals, may also be used to form a resonant standing wave in the cavity within cartridge 710 for resonant frequency-based distance/displacement measurement. In some implementations, a standing wave may be formed in the cartridge housing/walls (e.g., due to the different impedances of the cartridge housing/walls with and without contacting the medicine fluid), rather than in the medicine from cartridge 710, for determining a distance between piston 720 and the bottom 712 of cartridge 710.

Furthermore, as described above with respect to FIGS. 5 and 6, the resonant frequency-based distance/displacement measurement may be used to continuously measure the distance between dispensing piston 720 and the inner bottom 712 of cartridge 710 before, during, and/or after a drug dispensing. A change in measured distance between two measuring time instants may indicate the volume of the medicine being dispensed between the two measuring time instants, and hence the dispensing rate. A change in measured distance before and after a drug dispensing may indicate the total volume of the medicine being dispensed to the patient.

V. Time-of-Flight-Based Measurement

Another technique for distance/displacement measurement is to measure the time of flight (or round trip delay) for a wave signal (e.g., an acoustic signal or an electromagnetic wave signal, such as a light signal) to travel from a source to a target and then return from the target to the source (or a receiver near the source). The distance may therefore be given by the one-way time-of-flight multiplied by the speed of the wave signal traveling in the medium between the source and the target. Some time-of-flight sensors may operate by sending a series of short duration pulses to the target. Some time-of-flight sensors may use amplitude modulated continuous wave signals, and measure a phase shift in the modulation signal between the launched and the returned wave signals, and the time-of-flight may be determined by dividing the phase shift by the modulation frequency.

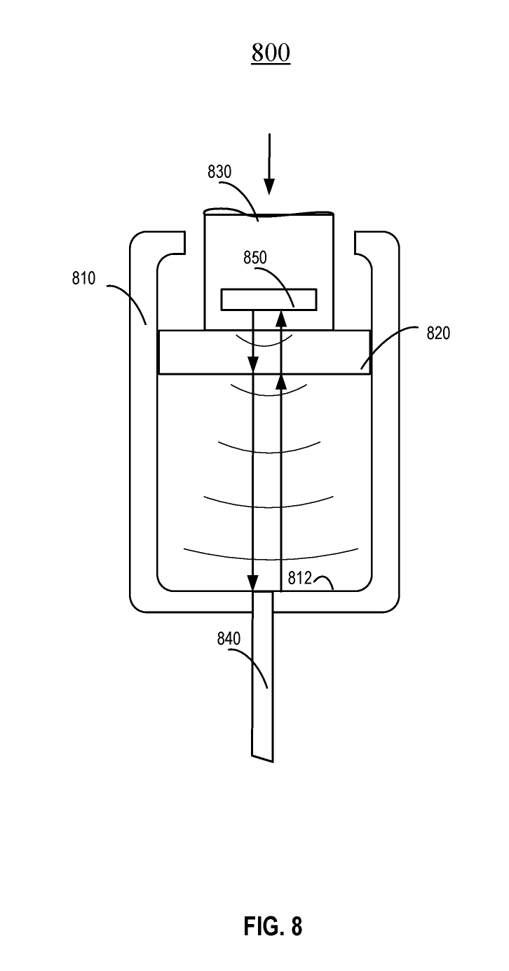

FIG. 8 illustrates an ultrasonic distance/displacement measurement unit based on time-of-flight measurement for determining the volume of a medicine in a medicine delivery device 800, according to certain embodiments. Medicine delivery device 800 may include a cartridge 810 containing a medicine to be dispensed to a patient, a dispensing piston 820 coupled to a shaft 830 (which may be coupled to a dose dispensing button not shown in FIG. 8), and a needle assembly 840. A user may push the dose dispensing button to dispense medicine from cartridge 810 to a patient through needle assembly 840.

Medicine delivery device 800 may also include an ultrasonic transceiver 850 located in dispensing piston 820 or shaft 830. Ultrasonic transceiver 850 may include a transmitter and a receiver. The transmitter may transmit ultrasonic pulses towards dispensing piston 820 and a bottom 812 of cartridge 810. At least a part of the transmitted ultrasonic pulses may be reflected at the interface between dispensing piston 820 and the liquid in cartridge 810 due to impedance mismatch. At least some of the transmitted ultrasonic pulses may travel through dispensing piston to the liquid in cartridge 810, and then travel through the liquid in cartridge 810 before reaching bottom 812 of cartridge 810. Due to the impedance mismatch between the liquid in cartridge 810 and bottom 812 of cartridge 810, some portions of the ultrasonic pulses that have reached bottom 812 of cartridge 810 may be reflected back towards ultrasonic transceiver 850. Similar transmission and reflection may be experienced by the ultrasonic pulses on the return path. The receiver may detect the returned ultrasonic pulses and determine a time of flight (or round-trip delay) of the ultrasonic pulses in the liquid in cartridge 810 based on the detected signals and the transmitted ultrasonic pulses. The length of the cavity in cartridge 810 that is filled with the liquid medicine may then be determined based on the speed of the ultrasonic pulses in the liquid medicine.

A person skilled in the art would appreciate that other signals, such as waves in other frequency bands, may also be used to measure the length of the cavity in cartridge 810 that is filled with the liquid medicine. It may be difficult to accurately measure the time of flight using waves (e.g., optical waves) that may have a high propagation speed in the liquid medicine due to the relatively short length of the cavity in cartridge 810.

Furthermore, as described above, the time-of-flight-based distance/displacement measurement may be used to continuously measure the distance between dispensing piston 820 and the inner bottom 812 of cartridge 810 before, during, and/or after a drug dispensing. A change in measured distance between two measuring time instants may indicate the volume of the medicine being dispensed between the two measuring time instants, and hence the dispensing rate. A change in measured distance before and after a drug dispensing may indicate the total volume of the medicine being dispensed to the patient.

VI. Frequency-Modulated Continuous-Wave (FM-CW) Time-of-Flight-Based Volume Measurement

As discussed above, it may be difficult to accurately measure the time of flight using waves (e.g., optical waves) that may have a high propagation speed in the liquid medicine due to the relatively short length of the cavity in the cartridge. A frequency-modulated continuous-wave (FM-CW) technique, also called a continuous-wave frequency-modulated (CWFM) technique, is a short-range distance measuring technique. In a FM-CW time-of-flight based system, the transmitted signal may be a continuous wave with a known stable frequency (center frequency or carrier frequency) modulated by a modulation signal such that the frequency of the continuous wave varies up and/or down over a period of time. The modulation signal may be, for example, a sine wave, a sawtooth wave, a triangle wave, a square wave, or other signals. The frequency difference between the received signal and the transmitted signal at a given time instant may increases with the delay between the received signal and the transmitted signal, and hence may increase with the distance that the continuous wave travels. Thus, the travelled distance may be determined by mixing the transmitted signal with the received signal to produce a beat signal (demodulation), and measuring the frequency of the beat signal that may represent the frequency difference between the transmitted signal and the received signal.

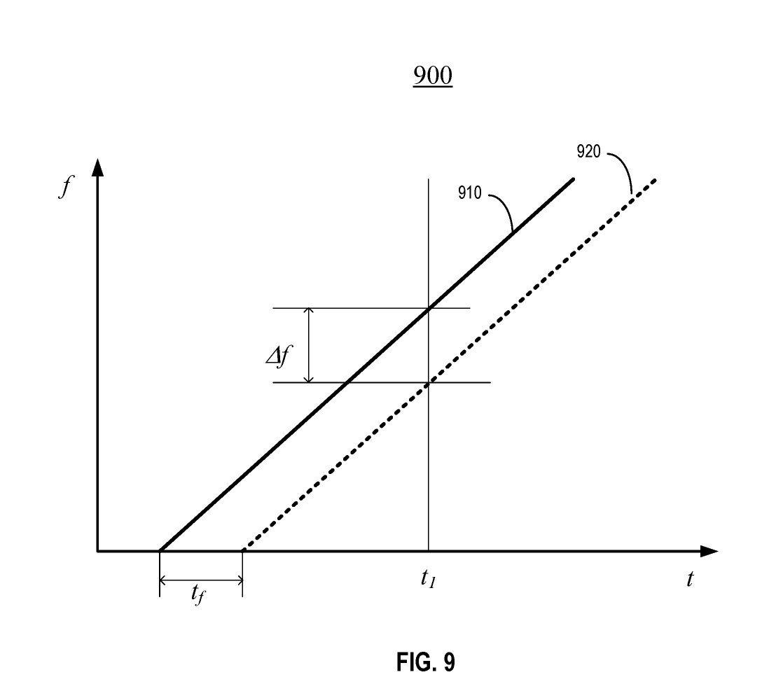

FIG. 9 is a diagram 900 illustrating time-of-flight-based short distance/displacement measurement using frequency modulated continuous wave, according to certain embodiments. In FIG. 9, a transmitted FM-CW wave 910 may be modulated by a sawtooth wave such that the frequency of the FM-CW wave has a linear chirp of frequency with time at a rate of df/dt. The returned wave 920 may also have a linear chirp of frequency with time at a rate of df/dt. However, because of the delay between the transmitted FM-CW wave and the received FM-CW wave, at any given time (e.g., time instant t1), the frequency of the transmitted FM-CW wave and the received FM-CW wave may be different by an amount of

$$Δ f = \frac{df}{dt} {t}_{f} ,$$

where t_{f} is the time of flight, as the received FM-CW wave at time instant t1 was generated at a time t_{f} before time instant t1. Therefore, when the transmitted wave and the received wave are combined at (i.e., detected by) a detector, a signal with the beat frequency of Δf may be generated by the detector. The beat frequency may then be determined by various known methods. Based on the beat frequency and the chirp rate

$$\frac{df}{dt} ,$$

the time of flight t_{f} may be determined, which may then be used to determine the length of the cavity in the cartridge as in the time-of-flight-based distance/displacement measurement technique described above with respect to FIG. 8.

It is noted that the above-described techniques for distance/displacement measurement are just some non-limiting examples. Other techniques, such as intensity-based techniques that may determine the length of the transmission path based on the attenuation of the transmitted signal, interferometer-based techniques, techniques using multiple wavelengths, techniques using optical fibers or fiber Bragg gratings, etc., may also be used to measure the dose of the medicine in a medicine delivery device or the dose of the medicine being dispensed from the medicine delivery device. For example, in some implementations, the electrical impedance of the cartridge housing (e.g., coated with a layer of conductive material) may depend on the area of the cartridge housing that is in contact with the medicine fluid, and thus may indicate the volume of the medicine in the cartridge. In some implementations, a pair of electrodes may be formed on the piston and the bottom of the cartridge, and a capacitance between the pair of electrodes may be measured to determine the distance between the piston and the bottom of the cartridge. In some implementations, a plurality of strain sensors distributed along the length of the cartridge may be used, and the total strains on the cartridge housing may be measured to determine the distance between the piston and the bottom of the cartridge.

VII. Example Method

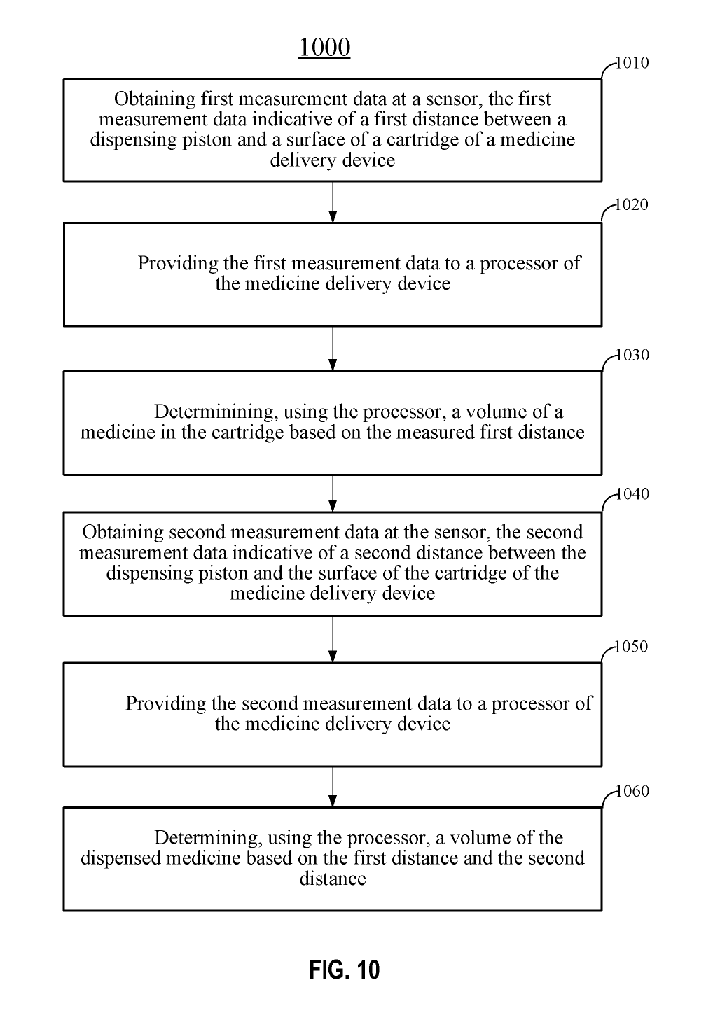

FIG. 10 is a simplified flow chart 1000 illustrating an example method of drug delivery measurement, according to certain embodiments. The process illustrated by flow chart 1000 may be performed by, for example, a computing system (e.g., hardware processor 312) of medicine delivery device 110, dose measurement unit 314, or various distance/displacement measurement units described above with respect to FIGS. 5-9.

At block 1010, a dose measurement unit (e.g., a distance/displacement measurement unit described above; as used herein, any reference to a dose measurement unit may be applicable to describe a distance measurement unit or distance measurement sensor) or a sensor may obtain first measurement data indicative of a first distance between a dispensing piston and a surface of a cartridge opposite the piston (e.g., a bottom surface of the cartridge if the piston is attached to a shaft at the top of the cartridge) in a medicine delivery device that stores a medicine before a drug dispensing is performed. As described above, the first distance may be measured using triangulation-based measurement techniques, resonant frequency-based measurement techniques, time-of-flight-based measurement techniques, FW-CW time-of-flight-based measurement techniques, or other suitable techniques, such as intensity-based measurement techniques. The first distance may be measured more than one time, invalid or erroneous results may be removed, and valid results may be averaged to determine the actual volume.

Means for performing the function of block 1010 may include, for example, the dose measurement unit 314 shown in FIG. 3.

At block 1020, the dose measurement unit or sensor may provide the first measurement data to a processor of the medicine delivery device.

Means for performing the function of block 1020 may include, for example, the dose measurement unit 314 shown in FIG. 3 and the hardware processor 312 shown in FIG. 3.

At block 1030, a processor in communication with the dose measurement unit may determine a volume of the medicine in the cartridge based on the measured first distance. For example, if the dimensions of the cartridge (e.g., the radius or diameter of the inner cavity of the cartridge) are known, the volume of the medicine in the cartridge may be determined by the first distance and the area of the cross-section of the inner cavity of the cartridge.

Means for performing the function of block 1030 may include, for example, the hardware processor 312 shown in FIG. 3.

At block 1040, during or after the drug dispensing, a dose measurement unit or a sensor may obtain second measurement data indicative of a second distance between the dispensing piston and the surface of the cartridge opposite the piston (e.g., a bottom surface of the cartridge if the piston is attached to a shaft at the top of the cartridge). may be measured by the dose measurement unit as described above with respect to block 1010. As described above, the second distance may be measured using triangulation-based measurement techniques, resonant frequency-based measurement techniques, time-of-flight-based measurement techniques, FW-CW time-of-flight-based measurement techniques, or other suitable techniques, such as intensity-based measurement techniques. The second distance may be measured more than one time, invalid or erroneous results may be removed, and valid results may be averaged to determine the actual volume.

Means for performing the function of block 1040 may include, for example, the dose measurement unit 314 shown in FIG. 3.

At block 1050, the dose measurement unit or sensor may provide the first measurement data to a processor of the medicine delivery device.

Means for performing the function of block 1050 may include, for example, the dose measurement unit 314 shown in FIG. 3 and the hardware processor 312 shown in FIG. 3.

At block 1060, the dose measurement unit or the computing system in communication with the dose measurement unit may determine a dispensing rate during the drug dispensing or a volume of the dispensed medicine based on the first distance and the second distance. For example, if the drug dispensing starts at time instant t1 and the second distance is measured at time instant t2, then the volume of the dispensed medicine may be determined based on the difference between the first distance and the second distance. The drug dispensing rate may be determined based on the volume of the dispensed medicine and the difference in time between time instant t1 and time instant t2.

Means for performing the function of block 1060 may include, for example, the hardware processor 312 shown in FIG. 3.

It is noted that even though FIG. 10 describes the operations as a sequential process, some of the operations may be performed in parallel or concurrently. In addition, the order of the operations may be rearranged. An operation may have additional steps not included in the figure. Some operations may be optional, and thus may be omitted in various embodiments. Some operations described in one block may be performed together with operations described at another block. Furthermore, embodiments of the methods may be implemented in hardware, software, firmware, middleware, microcode, hardware description languages, or any combination thereof.

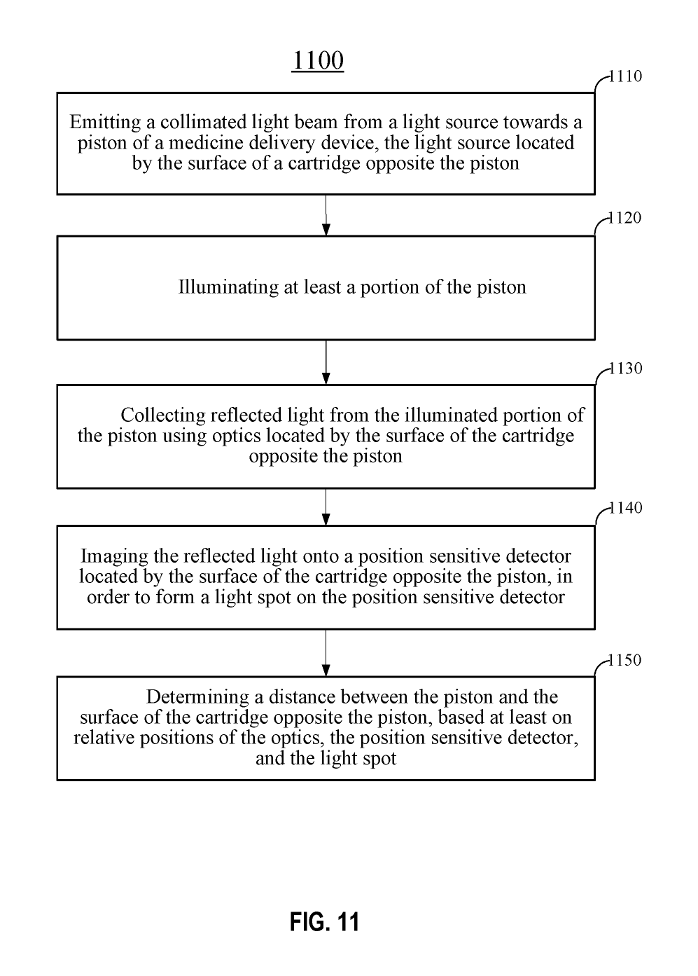

FIG. 11 is a simplified flow chart 1100 illustrating an example method of drug delivery measurement, according to certain embodiments. More specifically, the process illustrated by flow chart 1100 involves the use of an optical, triangulation-based method that can be performed by, for example, a computing system (e.g., hardware processor 312) of medicine delivery device 110, dose measurement unit 314, and/or an optical distance/displacement measurement unit/sensor as shown in FIG. 5.

At block 1110, a light source may emit a collimated light beam towards a piston of a medicine delivery device. The light source may be located by the surface of a cartridge opposite the piston. In various embodiments, the light source may be one of: a diode, an LED, or a laser.

Means for performing the function of block 1110 may include, for example, the light source 550 of the optical distance/displacement measurement unit/sensor shown in FIG. 5. The light source 550 may be controlled by a processor, such as hardware processor 312 shown in FIG. 3.

At block 1120, at least a portion of the piston may be illuminated (e.g., the collimated light beam emitted towards the piston will illuminate a portion of it).

Means for performing the function of block 1120 may also include the light emitted from the light source 550 of the optical distance/displacement measurement unit/sensor shown in FIG. 5, which may in turn be controlled by a processor, such as hardware processor 312 shown in FIG. 3.

At block 1130, reflected light from the illuminated portion of the piston may be collected using optics located by the surface of the cartridge opposite the piston.

Means for performing the function of block 1130 may include, for example, the optics 560 shown in FIG. 5.

At block 1140, the optics may image the reflected light onto a position sensitive detector by the surface of the cartridge opposite the piston. This will form a light spot on the position sensitive detector. The position sensitive detector may be an array of pixels configured to detect a position of the light spot. More specifically, the light spot will be on a portion of the position sensitive detector, and its location can be determined based on which of the pixels receive light.

Means for performing the function of block 1140 may include, for example, the optics 560 shown in FIG. 5.

At block 1150, the distance between the piston and the surface of the cartridge opposite the piston may be determined based at least on relative positions of the optics, the position sensitive detector, and the light spot. More specifically, the center of the light spot can be determined based on its location on the position sensitive detector. Then, a measured angle can be determined between a first line and a second line. The first line runs from the center of the optics to the center of the position sensitive detector and the second line runs from the center of the optics to the center of the light spot. The positions and centers of the optics and the position sensitive detector are known in advance and fixed. Afterwards, a distance can be determined between the light source and the optics. The positions and centers of the light source and the optics are also known in advance and fixed. With this information, the distance between the piston and the surface of the cartridge can be determined based on the measured angle and the distance between the light source and the optics. More information about this process is described in regards to FIG. 5.

The methods, systems, and devices discussed above are examples. Various configurations may omit, substitute, or add various procedures or components as appropriate. For instance, in alternative configurations, the methods may be performed in an order different from that described, and/or various stages may be added, omitted, and/or combined. Also, features described with respect to certain configurations may be combined in various other configurations. Different aspects and elements of the configurations may be combined in a similar manner. Also, technology evolves and, thus, many of the elements are examples and do not limit the scope of the disclosure or claims.

Terms “and,” “or,” and “an/or,” as used herein, may include a variety of meanings that also is expected to depend at least in part upon the context in which such terms are used. Typically, “or” if used to associate a list, such as A, B, or C, is intended to mean A, B, and C, here used in the inclusive sense, as well as A, B, or C, here used in the exclusive sense. In addition, the term “one or more” as used herein may be used to describe any feature, structure, or characteristic in the singular or may be used to describe some combination of features, structures, or characteristics. However, it should be noted that this is merely an illustrative example and claimed subject matter is not limited to this example. Furthermore, the term “at least one of” if used to associate a list, such as A, B, or C, may be interpreted to mean any combination of A, B, and/or C, such as A, B, C, AB, AC, BC, AA, AAB, AABBCCC, etc.

Reference throughout this specification to “one example,” “an example,” “certain examples,” or “exemplary implementation” means that a particular feature, structure, or characteristic described in connection with the feature and/or example may be included in at least one feature and/or example of claimed subject matter. Thus, the appearances of the phrase “in one example,” “an example,” “in certain examples,” “in certain implementations,” or other like phrases in various places throughout this specification are not necessarily all referring to the same feature, example, and/or limitation. Furthermore, the particular features, structures, or characteristics may be combined in one or more examples and/or features.

Specific details are given in the description to provide a thorough understanding of example configurations (including implementations). However, configurations may be practiced without these specific details. For example, well-known circuits, processes, algorithms, structures, and techniques have been shown without unnecessary detail in order to avoid obscuring the configurations. This description provides example configurations only, and does not limit the scope, applicability, or configurations of the claims. Rather, the preceding description of the configurations will provide those skilled in the art with an enabling description for implementing described techniques. Various changes may be made in the function and arrangement of elements without departing from the spirit or scope of the disclosure.

Also, configurations may be described as a process which is depicted as a flow diagram or block diagram. Although each may describe the operations as a sequential process, many of the operations may be performed in parallel or concurrently. In addition, the order of the operations may be rearranged. A process may have additional steps not included in the figure. Furthermore, examples of the methods may be implemented by hardware, software, firmware, middleware, microcode, hardware description languages, or any combination thereof. When implemented in software, firmware, middleware, or microcode, the program code or code segments to perform the necessary tasks may be stored in a non-transitory computer-readable medium such as a storage medium. Processors may perform the described tasks.

For an implementation involving firmware and/or software, the methodologies may be implemented with modules (e.g., procedures, functions, and so on) that perform the functions described herein. Any machine-readable medium tangibly embodying instructions may be used in implementing the methodologies described herein. For example, software codes may be stored in a memory and executed by a processor unit. Memory may be implemented within the processor unit or external to the processor unit. As used herein the term “memory” refers to any type of long term, short term, volatile, nonvolatile, or other memory and is not to be limited to any particular type of memory or number of memories, or type of media upon which memory is stored.

If implemented in firmware and/or software, the functions may be stored as one or more instructions or code on a computer-readable storage medium. Examples include computer-readable media encoded with a data structure and computer-readable media encoded with a computer program. Computer-readable media includes physical computer storage media. A storage medium may be any available medium that may be accessed by a computer. By way of example, and not limitation, such computer-readable media may comprise RAM, ROM, EEPROM, compact disc read-only memory (CD-ROM) or other optical disk storage, magnetic disk storage, semiconductor storage, or other storage devices, or any other medium that may be used to store desired program code in the form of instructions or data structures and that may be accessed by a computer; disk and disc, as used herein, includes compact disc (CD), laser disc, optical disc, digital versatile disc (DVD), floppy disk and blu-ray disc where disks usually reproduce data magnetically, while discs reproduce data optically with lasers. Combinations of the above should also be included within the scope of computer-readable media.

In addition to storage on computer-readable storage medium, instructions and/or data may be provided as signals on transmission media included in a communication apparatus. For example, a communication apparatus may include a transceiver having signals indicative of instructions and data. The instructions and data are configured to cause one or more processors to implement the functions outlined in the claims. That is, the communication apparatus includes transmission media with signals indicative of information to perform disclosed functions. At a first time, the transmission media included in the communication apparatus may include a first portion of the information to perform the disclosed functions, while at a second time the transmission media included in the communication apparatus may include a second portion of the information to perform the disclosed functions

Having described several example configurations, various modifications, alternative constructions, and equivalents may be used without departing from the spirit of the disclosure. For example, the above elements may be components of a larger system, wherein other rules may take precedence over or otherwise modify the application of the invention. Also, a number of steps may be undertaken before, during, or after the above elements are considered.

What is claimed is:

1. A medicine delivery device comprising:
a shaft attached to a piston, wherein the shaft is configured to push the piston through a cartridge storing medicine to dispense the medicine from the cartridge through a needle assembly; and
an optical distance measuring sensor configured to measure a distance between the piston and a surface of the cartridge opposite the piston.

2. The device of claim 1, wherein the optical distance measuring sensor comprises a light source, optics, and a position sensitive detector.

3. The device of claim 2, wherein the position sensitive detector is an array of pixels configured to detect light.

4. The device of claim 1, wherein the optical distance measuring sensor is located at the bottom of the cartridge.

5. The device of claim 2, wherein the light source comprises one of: a diode, an LED, or a laser, and wherein the light source is configured to emit a light beam.

6. The device of claim 5, wherein the light beam is collimated and sent towards the piston to illuminate at least a portion of the piston.

7. The device of claim 6, wherein the optics are configured to collect reflected light from the illuminated portion of the piston and image the reflected light onto the position sensitive detector.

8. The device of claim 7, wherein the reflected light from the illuminated portion of the piston forms a light spot on the position sensitive detector, and wherein the position sensitive detector is an array of pixels configured to detect a position of the light spot.

9. The device of claim 8, wherein a triangle is formed by a first line from the center of the optics to the center of the position sensitive detector and a second line from the center of the optics to the center of the light spot.

10. The device of claim 9, wherein the distance between the piston and the surface of the cartridge opposite the piston can be determined based on a distance between the light source and the piston, and wherein the distance between the light source and the piston can be determined based at least on:
a measured angle between the first line and the second line; and
a distance between the light source and the optics.

11. A method for measuring a distance between a piston of a medicine delivery device and a surface of a cartridge opposite the piston, the method comprising:
emitting a collimated light beam from a light source towards the piston, wherein the light source is located by the surface of the cartridge opposite the piston;
illuminating at least a portion of the piston;
collecting reflected light from the illuminated portion of the piston using optics located by the surface of the cartridge opposite the piston;
imaging, using the optics, the reflected light onto a position sensitive detector by the surface of the cartridge opposite the piston, in order to form a light spot on the position sensitive detector; and
determining a distance between the piston and the surface of the cartridge opposite the piston based at least on relative positions of the optics, the position sensitive detector, and the light spot.

12. The method of claim 11, wherein the position sensitive detector is an array of pixels configured to detect light.

13. The method of claim 11, wherein the surface of the cartridge opposite the piston is the bottom of the cartridge.

14. The method of claim 11, wherein the light source comprises one of: a diode, an LED, or a laser.

15. The method of claim 11, wherein the position sensitive detector is an array of pixels configured to detect a position of the light spot.

16. The method of claim 11, wherein determining a distance between the piston and the surface of the cartridge opposite the piston further comprises:
determining a center of the light spot; and
determining a measured angle between a first line and a second line, wherein the first line is from the center of the optics to the center of the position sensitive detector, and wherein the second line is from the center of the optics to the center of the light spot.

17. The method of claim 16, wherein determining a distance between the piston and the surface of the cartridge opposite the piston further comprises:
determining a distance between the light source and the optics;
determining the distance between the piston and the surface of the cartridge based on the measured angle and the distance between the light source and the optics.

18. The method of claim 11, wherein the light beam is in the infrared spectrum band.

19. The method of claim 11, wherein the optics is a lens.

20. The method of claim 11, wherein the emitted light beam is perpendicular to the surface of the cartridge opposite the piston.

21. A medicine delivery device comprising:
means for emitting a collimated light beam towards a piston, wherein the piston is configured to be pushed through a cartridge storing medicine to dispense the medicine from the cartridge;
means for illuminating at least a portion of the piston;
means for collecting reflected light from the illuminated portion of the piston at a location by the surface of the cartridge opposite the piston;
means for imaging the reflected light into a light spot;
means for determining the location of the light spot; and
means for determining a distance between the piston and the surface of the cartridge opposite the piston based at least on the location of the light spot.

22. The medicine delivery device of claim 21, further comprising:
means for determining a center of the light spot.

23. The medicine delivery device of claim 22, wherein the light beam is in the infrared spectrum band.

24. The medicine delivery device of claim 22, wherein the emitted light beam is perpendicular to the surface of the cartridge opposite the piston.

25. A non-transitory computer readable medium containing instructions that, when executed by a processor, cause the processor to:
cause a light source to emit a collimated light beam from the light source towards a piston of a medicine delivery device, wherein the light source is located by a surface of a cartridge opposite the piston, wherein the cartridge stores medicine;
cause at least a portion of the piston to be illuminated;
cause reflected light from the illuminated portion of the piston to be collected using optics located by the surface of the cartridge opposite the piston;
cause the optics to image the reflected light onto a position sensitive detector by the surface of the cartridge opposite the piston, in order to form a light spot on the position sensitive detector; and
determine a distance between the piston and the surface of the cartridge opposite the piston based at least on relative positions of the optics, the position sensitive detector, and the light spot.

26. The non-transitory computer readable medium of claim 25, wherein the instructions, when executed by a processor, further cause the processor to:
determine a center of the light spot from a position of the light spot on the position sensitive detector;
determine a center of the position sensitive detector; and
determine a center of the optics.

27. The non-transitory computer readable medium of claim 26, wherein the instructions, when executed by a processor, further cause the processor to:
determine a measured angle between a first line and a second line, wherein the first line runs from the center of the optics to the center of the position sensitive detector, and wherein the second line runs from the center of the optics to the center of the light spot.

28. The non-transitory computer readable medium of claim 27, wherein the instructions, when executed by a processor, further cause the processor to:
determine a distance between the light source and the optics.

29. The non-transitory computer readable medium of claim 28, wherein the instructions, when executed by a processor, further cause the processor to:
determine a distance between the light source and the piston based at least on:
the measured angle between the first line and the second line; and
the distance between the light source and the optics.

30. The non-transitory computer readable medium of claim 29, wherein the instructions, when executed by a processor, further cause the processor to:
determine the distance between the piston and the surface of the cartridge opposite the piston based at least on the distance between the light source and the piston.
